(19) United States
(12) Patent Application Publication
(10) Pub. No.: US 2007/0029185 A1
(43) Pub. Date: Feb. 8, 2007

(54) Apparatus for producing nanoparticles

(75) Inventor: Tsai-Shih Tung, Tu-Cheng (TW)

Correspondence Address: PCE INDUSTRY, INC.;ATT. CHENG-JU CHIANG JEFFREY T. KNAPP, 458 E. LAMBERT ROAD, FULLERTON, CA, 92835, US

(73) Assignee: HON HAI Precision Industry CO., LTD., Tu-Cheng City (TW)

(21) Appl. No.: 11/377,850

(22) Filed: Mar. 16, 2006

(30) Foreign Application Priority Data
Aug. 8, 2005 (CN) 200510036491.6

(51) Int. Cl. B01J 19/12; A61K 9/14

(52) U.S. Cl. 204/157.41; 424/489; 977/906

(57) ABSTRACT

An apparatus for producing nanoparticles includes a vessel, a target, and a laser generating device. The vessel has an inlet and an outlet. The target is configured on a bottom of the vessel and disposed below the inlet. The produced nanoparticles flow continuously from the outlet with the produced solution and the protective solution can also be supplied into the vessel continuously from the inlet during production so as to mass-produce nanoparticles in a continuous and high efficient manner.

### BACKGROUND

1. Technical Field

The present invention relates to apparatuses for producing nanoparticles, and particularly to an apparatus for producing nanoparticles by a laser ablation method.

2. Related Art

Nanoparticles are small clusters of atoms about 1 to 100 nm length and are an increasingly important industrial material. Due in part to their high surface area and high reactivity, nanoparticles may be used in a variety of applications, such as semiconductor technology, magnetic storage, electronics fabrication, reaction catalysis, and drug delivery. The extremely large ratio of surface area to weight allows nanoparticles to interact with their surroundings very quickly in a way that can also lead to the fabrication of new materials having new properties.

Laser ablation method is one of the methods for producing nanoparticles. The method includes the steps of

(1) placing a block of highly pure material in a vessel;

(2) immersing the material in the vessel with a protective solution with a proper amount of stabilizing agent;

(3) bombarding the material with a high power laser beam to ablate the material to generate nanoparticles; and

(4) dispersing the produced nanoparticles uniformly throughout the protective solution.

The stabilizing agent in the protective solution, such as surfactants, can adhere to surface of the nanoparticles to generate static electric force. This prevents the nanoparticles from agglomerating together.

A general apparatus for producing nanoparticles with laser ablation includes a vessel, such as beaker, adapted for containing a protective solution such as surfactants, a block of material, such as gold plate, configured on a bottom of the vessel, and a laser generating device adapted for generating a laser beam to bombard the material to produce nanoparticles.

A process for producing nanoparticles with above-described apparatus includes the steps of:

(1) placing a block of material in the vessel;

(2) supplying a protective solution into the vessel to immerse the material completely;

(3) bombarding the material with a high power laser beam generated by the laser generating device to ablate the material to generate nanoparticles;

(4) dispersing the produced nanoparticles in the protective solution uniformly; and

(5) collecting the solution containing the nanoparticles after a concentration of nanoparticles in the protective solution reaches a predetermined value.

Such a producing process has the following disadvantages. The two actions of supplying and collecting cannot be preformed simultaneously Therefore, protective solution must once again be added into the vessel to continue the production after collecting the produced solution. Thus it can be seen that this method is not suited to continuous mass, and also that the efficiency of nanoparticle production is very low.

What is needed, therefore, is an apparatus capable of high efficiency continuous production of nanoparticles.

### SUMMARY

An exemplary embodiment of the present apparatus for continuous production of nanoparticles is provided.

The apparatus includes a vessel containing a solution of surfactant, a target disposed below the inlet in the vessel; and a laser generating device for applying a laser beam to the target. The vessel has an inlet and an outlet.

Preferably, an ultrasonic device is configured for dispersing the nanoparticles evenly throughout the solution.

### BRIEF DESCRIPTION OF THE DRAWINGS

The above-mentioned and other features and advantages of the present apparatus will become more apparent and the invention will be better understood by reference to the following description of embodiments thereof taken in conjunction with the accompanying drawings.

FIG. 1 is a schematic view of an apparatus for producing nanoparticles in accordance with a first embodiment of the present invention;

FIG. 2 is similar to FIG. 1, but showing an apparatus for producing nanoparticles with an ultrasonic device configured on a bottom of the vessel, in accordance with a second embodiment of the present invention; and

FIG. 3 is similar to FIG. 2, but showing an apparatus for producing nanoparticles in accordance with a third embodiment of the present invention.

Corresponding reference characters indicate corresponding parts throughout the several views. The exemplifications set out herein illustrate at least one preferred embodiment of the invention, in one form, and such exemplifications are not to be construed as limiting the scope of the invention in any manner.

### DETAILED DESCRIPTION OF PREFERRED EMBODIMENTS

Reference will now be made to the drawings to describe in detail the preferred embodiments of the present apparatus for producing nanoparticles.

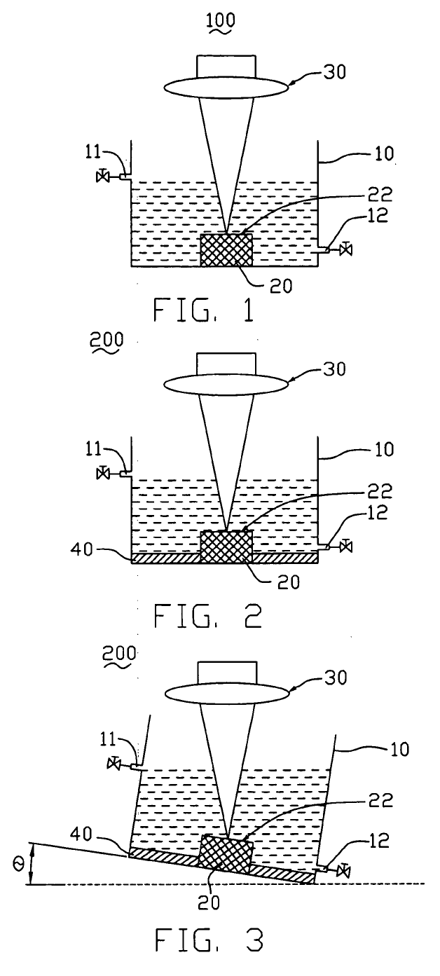

Referring to FIG. 1, an apparatus 100 for producing nanoparticles according to a first embodiment of the present invention is shown. The apparatus 100 includes a vessel 10, a target 20, and a laser generating device 30.

The vessel 10 is adapted for containing a protective solution of surfactant, and has an inlet 11 and an outlet 12. Two valves are connected with the inlet 11 and outlet 12 respectively. The inlet 11 is adapted for supplying the protective solution into the vessel 10, and configured in an upside of a sidewall of the vessel 10. The outlet 12 is adapted for collecting a produced solution containing nanoparticles, and configured in the same or another sidewall of the vessel 10 and adjacent to a bottom of the vessel 10.

The target 20 is configured on a bottom of the vessel 10 and has a top surface 22. The target 20 is disposed below the inlet 11 and the top surface 22 of the target 20 is above the outlet 12. The target 20 can be comprised of a material selected from the group consisting of carbon, metal carbide, metal and metal alloy.

The laser generating device 30 is adapted for generating a laser beam to bombard the target 20. The laser generating device 30 can be selected from the group consisting of a gas laser, a liquid laser, a solid laser, and a semiconductor laser.

Referring to FIG. 2, an apparatus 200 for producing nanoparticles according to a second embodiment is shown. The apparatus 200 includes a vessel 10 having an inlet 11 and an outlet 12, a target 20 having a top surface 22 configured on a bottom of the vessel 10, and a laser generating device 30 adapted for generating a laser beam to bombard the target 20. The inlet 11 is configured in an upside of a sidewall of the vessel 10. The outlet 12 is configured in the same or another sidewall of the vessel 10 and adjacent to the bottom of the vessel 10. The target 20 is disposed below the inlet 11 and the top surface 22 of the target 20 is above the outlet 12.

In addition, the apparatus 200 includes an ultrasonic device 40. The ultrasonic device 40 is configured on the bottom of the vessel 10 in order to make the produced nanoparticles disperse throughout the protective solution more uniformly.

Referring to FIG. 3, an explanatory view for explaining the operation of the apparatus 200 for producing nanoparticles according to a third embodiment is shown. The vessel 10 can be placed at an angle θ with respect to the horizontal during production in order to supply the protective solution into the vessel 10 and collect the produced solution containing the nanoparticles more conveniently, wherein the angle θ satisfies the following formulation: 0≦θ≦60°. A process for producing nanoparticles with above-described apparatus 200 is described in detail as follows:

(1) A target 20 is placed on a bottom of the vessel 10. The target 20 is made of copper in the present embodiment;

(2) A protective solution is supplied into the vessel 10 to immerse the target 20 in the protective solution completely. The protective solution can be an aqueous solution of polyvinyl alcohol, polyoxyethylene nonyl phenyl ether, alkyl phenol polyoxyethylene ether, and sodium dodecyl sulfonate. In the present embodiment, the protective liquid is an aqueous solution of polyvinyl alcohol;

(3) A laser beam generated by the laser generating device 30 is applied to the target 20 thereby bombarding the target 20. The laser generating device 30 is a solid-state laser in the present embodiment;

(4) The produced nanoparticles are dispersed throughout the protective solution uniformly using an ultrasound generated by the ultrasonic device 40;

(5) The produced solution containing the nanoparticles is discharged via the outlet and collected after a concentration of nanoparticles in the solution reaches a predetermined value. The protective solution is then supplied into the vessel 10 from the inlet 11 to realize the continuous production of nanoparticles.

An advantage of the apparatus 200 is that the produced nanoparticle solution can continually flow out from the outlet 12 whilst the protective solution is simultaneously supplied into the vessel 10 from the inlet 11 during nanoparticle production so as to mass-produce nanoparticles in a continuous and high efficient manner.

While the present invention has been described as having preferred or exemplary embodiments, the embodiments can be further modified within the spirit and scope of this disclosure. This application is therefore intended to cover any variations, uses, or adaptations of the embodiments using the general principles of the invention as claimed. Further, this application is intended to cover such departures from the present disclosure as come within known or customary practice in the art to which the invention pertains and which fall within the limits of the appended claims or equivalents thereof.

What is claimed is:

1. An apparatus for producing nanoparticles, comprising:
a vessel for containing a solution of surfactant, the vessel having an inlet and an outlet;
a target disposed below the inlet in the vessel; and
a laser generating device for applying a laser beam to the target.

2. The apparatus as described in claim 1, further comprising an ultrasonic device configured for dispersing the nanoparticles in the solution.

3. The apparatus as described in claim 1, wherein the target is comprised of a material selected from the group consisting of carbon, metal carbide, metal and metal alloy.

4. The apparatus as described in claim 3, wherein the target is comprised of a material selected from the group consisting of copper, silver, gold and any combination alloy thereof.

5. The apparatus as described in claim 1, wherein the laser generating device is selected from the group consisting of a gas laser, a liquid laser, a solid-state laser, and a semiconductor laser.

6. The apparatus as described in claim 1, wherein the outlet is configured to be lower than a top surface of the target.

7. The apparatus as described in claim 1, wherein the vessel is placed at an angle θ with respect to horizontal, and the angle θ satisfies the following formulation: 0≦θ≦60°.
